(19) United States
(12) Patent Application Publication
(10) Pub. No.: US 2024/0180513 A1
(43) Pub. Date: Jun. 6, 2024

(54) METHOD AND SYSTEM FOR MONITORING REGURGITATION

(71) Applicant: WUHAN UNITED IMAGING HEALTHCARE CO., LTD., Wuhan (CN)

(72) Inventors: Ming ZHOU, Wuhan (CN); Man WANG, Wuhan (CN)

(73) Assignee: WUHAN UNITED IMAGING HEALTHCARE CO., LTD., Wuhan (CN)

(21) Appl. No.: 18/525,882

(22) Filed: Dec. 1, 2023

(30) Foreign Application Priority Data
Dec. 1, 2022 (CN) 202211531694.2

(51) Int. Cl. A61B 8/06; A61B 8/08

(52) U.S. Cl. CPC A61B 8/06; A61B 8/0883; A61B 8/488

(57) ABSTRACT

The embodiment of the present disclosure discloses a method and system for monitoring regurgitation. The method includes: determining regurgitation information of a plurality of color Doppler images based on color Doppler data of a target position; and generating a regurgitation information data graph based on the regurgitation information of the plurality of color Doppler images. The regurgitation information data graph may reflect a correspondence between at least one frame number of at least one of the plurality of color Doppler images and the regurgitation information of the at least one color Doppler image.

### CROSS-REFERENCE TO RELATED APPLICATION

This application claims priority of Chinese Patent Application No. 202211531694.2 filled on Dec. 1, 2022, the content of which is entirely incorporated herein by reference.

### TECHNICAL FIELD

The present disclosure relates to a field of medical technology, in particular to methods and systems for monitoring regurgitation.

### BACKGROUND

Existing methods usually directly observe a heart valve regurgitation with naked eyes. However, observing a heart valve regurgitation is a qualitative diagnostic process, and quantitative values are greatly affected by factors such as an operator subjectivity, a speed range, an intracardiac pressure and a heart volume, etc. In addition, for inexperienced doctors or a pathological regurgitation with a small regurgitation flow, a misdiagnosis may occur.

Thus, there is an urgent need for real-time, automatic, and effective methods for monitoring regurgitation of the heart valve.

### SUMMARY

One of the embodiments of the present disclosure provides a system for monitoring regurgitation including: a determination module configured to determine the regurgitation information of a plurality of color Doppler images based on color Doppler data of a target position; and a generating module configured to generate a regurgitation information data graph based on the regurgitation information of the plurality of color Doppler images, and the regurgitation information data graph may reflect a correspondence between at least one frame number of at least one of the plurality of color Doppler images and the regurgitation information of the at least one color Doppler image.

In some embodiments, wherein the operations further comprise: determining, a regurgitation degree of the target position based on the regurgitation information data graph.

In some embodiments, wherein determining, the the regurgitation degree of the target position based on the regurgitation information data graph comprises: determining, the regurgitation degree of the target position based on a maximum value, an average value, or a weighted value of the regurgitation information in the regurgitation information data graph.

In some embodiments, wherein the determining, the regurgitation degree of the target position based on the regurgitation information data graph comprises: determining, a length of a regurgitation region of the target position based on the regurgitation information of the plurality of color Doppler images; and determining, the regurgitation degree of the target position based on a ratio of the length of the regurgitation region of the target position to a length of the target position along a regurgitation direction.

In some embodiments, wherein determining, the the regurgitation degree of the target position based on the ratio of the length of the regurgitation region of the target position to the length of the target position along the regurgitation direction comprises: in response to determining that a ratio of the length of the regurgitation region of the target position to the length of the target position along the regurgitation direction is less than ⅓, determining, that the regurgitation degree of the target position is a mild regurgitation; in response to determining that the ratio value of the length of the regurgitation region of the target position to the length of the target position along the regurgitation direction is in a range of ⅓-⅔, determining, that the regurgitation degree of the target position is a moderate regurgitation; and in response to determining that the ratio value of the length of the regurgitation region of the target position to the length of the target position along the regurgitation direction is greater than ⅔, determining, that the regurgitation degree of the target position is a severe regurgitation.

In some embodiments, wherein the determining, the regurgitation degree of the target position based on the regurgitation information data graph comprises:

determining, a width of a regurgitation region of the target position based on the regurgitation information of the plurality of color Doppler images; and determining, the regurgitation degree of the target position based on a ratio of the width of the regurgitation region of the target position to an inner diameter of the target position.

In some embodiments, wherein the determining, the regurgitation degree of the target position based on the ratio of the width of the regurgitation region of the target position to the inner diameter of the target position comprises: in response to determining that the ratio of the width of the regurgitation region of the target position to the inner diameter of the target position is in a range of 20%-40%, determining, that the regurgitation degree of the target position is a mild regurgitation; in response to determining that the ratio of the width of the regurgitation region of the target position to the inner diameter of the target position is in a range of 40%-60%, determining, that the regurgitation degree of the target position is a moderate regurgitation; or in response to determining that the ratio of the width of the regurgitation region of the target position to the inner diameter of the target position is greater than 60%, determining, that the regurgitation degree of the target position is a severe regurgitation.

In some embodiments, wherein the determining, the regurgitation degree of the target position based on the regurgitation information data graph comprises: determining, an area of a regurgitation region of the target position based on the regurgitation information of the plurality of color Doppler images; and determining, the regurgitation degree of the target position based on the area of the regurgitation region of the target position and an area of the target position.

In some embodiments, wherein the determining, the regurgitation degree of the target position based on the area of the regurgitation region of the target position and the area of the target position comprises: in response to determining that the area of the regurgitation region is less than 3 cm² or a ratio of the area of the regurgitation region to the area of the target position is less than 20%, determining, that the regurgitation degree of the target position is a mild regurgitation; in response to determining that the area of the regurgitation region is in the range of 3 cm²-4.5 cm² or the ratio of the area of the regurgitation region to the area of the target position being in the range of 20%-50%, determining, that the regurgitation degree of the target position is a moderate regurgitation; and in response to determining that the area of the regurgitation region is greater than 4.5 cm² or the ratio of the area of the regurgitation region to the area of the target position is greater than 50%, determining, that the regurgitation degree of the target position is a severe regurgitation.

In some embodiments, wherein the determining, the regurgitation degree of the target position based on the regurgitation information data graph comprises: determining, a regurgitation speed of the target position based on the regurgitation information of the plurality of color Doppler images; and determining, the regurgitation degree of the target position based on the regurgitation speed.

In some embodiments, wherein the determining, the regurgitation degree of the target position based on the regurgitation speed comprises: in response to determining that the regurgitation speed is less than 150 cm/s, determining, that the regurgitation degree of the target position is a mild regurgitation; in response to determining that the regurgitation speed is in a range of 150 cm/s-450 cm/s, determining, that the regurgitation degree of the target position is a moderate regurgitation; and in response to determining that the regurgitation speed is greater than 450 cm/s, determining, that the regurgitation degree of the target position a severe regurgitation.

In some embodiments, wherein, the operations further comprise: generating, a regurgitation warning signal based on the regurgitation degree of the target position.

In some embodiments, wherein the operations further comprise: displaying, a color Doppler image of the target position according to an interaction between a user and the regurgitation information data graph, regurgitation information of the color Doppler image being displayed on the color Doppler image.

In some embodiments, wherein the operations further comprise: displaying, one of the at least one of the plurality of color Doppler images and the regurgitation information data graph simultaneously in the user interface.

In some embodiments, wherein the regurgitation information comprises at least one of: a length of a regurgitation region, a width of the regurgitation region, an area of the regurgitation region, a direction of blood flow in the regurgitation region, or a speed of blood flow in the regurgitation region.

In some embodiments, wherein the regurgitation information data graph is obtained based on one or more color Doppler images selected from the plurality of color Doppler images, in each of the one or more color Doppler images, the regurgitation information is greater than a threshold.

In some embodiments, wherein the target position includes at least one of: a tricuspid valve, a mitral valve, or an aortic valve.

One of the embodiments of the present disclosure provides a method for monitoring regurgitation including: determining, regurgitation information of a plurality of color Doppler images based on color Doppler data of a target position; and generating a regurgitation information data graph based on the regurgitation information of the plurality of color Doppler images, the regurgitation information data graph reflecting a correspondence between at least one frame number of at least one of the plurality of color Doppler images and the regurgitation information of the at least one color Doppler image.

In some embodiments, the method further comprising: determining, a regurgitation degree of the target position based on the regurgitation information data graph.

One of the embodiments of the present disclosure provides a device for monitoring regurgitation, including a processor configured to execute the method for monitoring regurgitation.

One of the embodiments of the present disclosure provides a non-transitory computer-readable storage medium, the storage medium storing computer instructions, and after the computer reads the computer instructions in the storage medium, the computer executes the method for monitoring regurgitation.

Some embodiments of the present disclosure calculate a region of interest (ROI) of the regurgitation in the image frame through the color Doppler data, track the blood flow by updating the position of the blood flow detected in the ROI, thereby calculating the regurgitation region data to achieve a quantitative analysis.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an application scenario of an exemplary system for monitoring a regurgitation according to some embodiments of the present disclosure;

FIG. 2 is a block diagram illustrating an exemplary system for monitoring a regurgitation according to some embodiments of the present disclosure;

FIG. 3 is a flow chart illustrating an exemplary method for monitoring a regurgitation according to some embodiments of the present disclosure;

FIG. 4 is a schematic diagram illustrating a mitral valve regurgitation based on other embodiments of the present disclosure;

FIG. 5 is a schematic diagram illustrating an exemplary ultrasound imaging operation interface according to some embodiments of the present disclosure; and

FIG. 6 is a schematic diagram illustrating an exemplary ultrasound imaging operation interface according to some embodiments of the present disclosure.

### DETAILED DESCRIPTION

In order to more clearly illustrate the technical solutions of the embodiments of the present disclosure, the following will briefly introduce the drawings that need to be used in the description of the embodiments. Obviously, the drawings in the following description are only some examples or embodiments of the present disclosure, and those skilled in the art may apply this present disclosure to other similar situations based on these drawings and on the premise of not paying creative labor. Unless obviously obtained from the context or the context illustrates otherwise, the same numeral in the drawings refers to the same structure or operation.

It will be understood that the terms “system,” “device,” “unit,” and/or “module” used herein are one method to distinguish different components, elements, parts, sections, or assemblies of different levels. However, if other words may achieve the same purpose, the words may be replaced by other expressions.

The terminology used herein is for the purposes of describing particular examples and embodiments only and is not intended to be limiting. As used herein, the singular forms “a,” “an,” and “the” may be intended to include the plural forms as well, unless the context clearly indicates otherwise. In general, the terms “comprise,” and “include,” merely prompt to include steps and elements that have been clearly identified, and these steps and elements do not constitute an exclusive listing. The methods or devices may also include other steps or elements.

Flowcharts are used in this disclosure to illustrate operations performed by the system based on the embodiments of the present disclosure. It should be understood that the preceding or following operations of the flowchart may not necessarily be implemented in order. Instead, the operation may be processed in reverse order or simultaneously. Moreover, one or more other operations may be added into the flowcharts. One or more operations may be removed from the flowcharts.

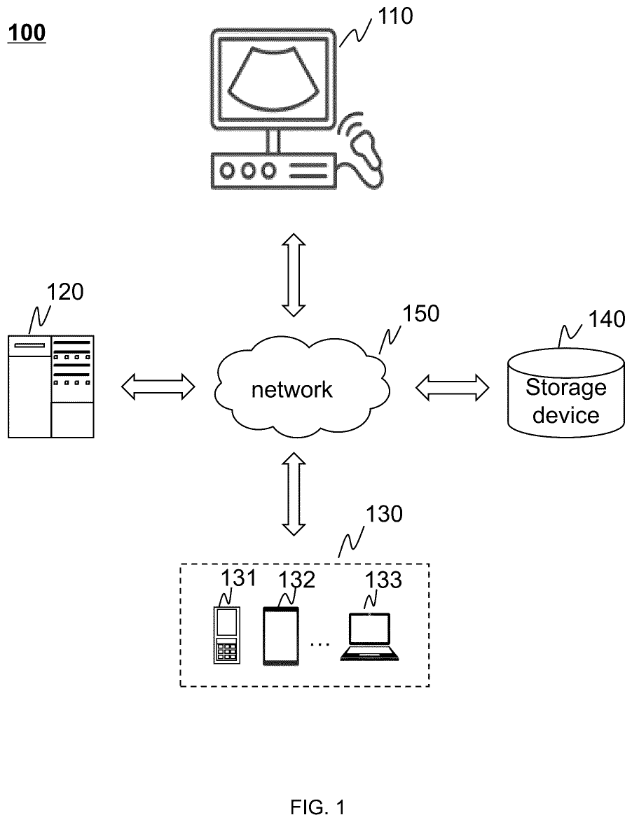

FIG. 1 is a schematic diagram illustrating an application scenario of an exemplary system for monitoring a regurgitation according to some embodiments of the present disclosure.

As shown in FIG. 1, an application scenario 100 of a monitoring system for the regurgitation may include an ultrasound device 110, a processing device 120, a terminal device 130, a storage device 140, and a network 150.

The ultrasound device 110 refers to a device that utilizes a propagation law of ultrasonic waves in a medium to obtain an image or a video of an object. For example, the ultrasound device 110 may include an ultrasonic pulse echo imaging device, an ultrasonic echo Doppler imaging device, an ultrasonic electronic endoscope, an ultrasonic Doppler blood flow analysis device, an ultrasonic human tissue measurement device, etc. The object is an object to be imaged, e.g., a patient, and a wounded, etc. In some embodiments, the object may be ultrasound examined in any position (e.g., a supine position, a lateral position, a prone position, a semi-recumbent position, or a sitting position). In some embodiments, a scanning mode of the ultrasound device 110 may include an amplitude modulated ultrasound (A-ultrasound), a brightness modulated ultrasound (B-ultrasound), an ultrasonic spot scanning (M-ultrasound), and a Doppler ultrasound (D-ultrasound), etc. In some embodiments, the ultrasound device 110 may be disposed in a medical place or a medical facility, such as a ward, a delivery room, an examination room, an operation room, a first-aid room, an ambulance, etc. In some embodiments, the ultrasound device 110 may transmit ultrasonic data to the processing device 120 through the network 150. A relevant description of the above ultrasound device 110 is for illustration purposes only, and is not intended to limit the scope of the present disclosure.

The processing device 120 may process data and/or information obtained from the ultrasound device 110, the terminal device 130, the storage device 140 and/or other components of the application scenario 100 of the monitoring system. For example, the processing device 120 may obtain the ultrasonic data from the ultrasound device 110, the terminal device 130, and the storage device 140, and analyze and process the obtained ultrasonic data.

In some embodiments, the processing device 120 may be a single server or a server group. The server group may be centralized or distributed. In some embodiments, the processing device 120 may be local or remote. For example, the processing device 120 may access information and/or data from the ultrasound device 110, the terminal device 130 and/or the storage device 140 through the network 150. For another example, the processing device 120 may be directly connected to the ultrasound device 110, the terminal device 130 and/or the storage device 140 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, etc., or any combination thereof.

In some embodiments, the processing device 120 and the ultrasound device 110 may be integrated. In some embodiments, the processing device 120 and the ultrasound device 110 may be connected directly or indirectly, and work together to implement the methods and/or functions described in the present disclosure.

In some embodiments, the processing device 120 may include an input device and/or an output device. Through the input device and/or the output device, an interaction with the user (e.g., the interaction with a regurgitation information data graph, etc.) may be implemented. In some embodiments, the input device and/or the output device may include a display screen, a keyboard, a mouse, a microphone, a trackball, etc., or any combination thereof.

The terminal device 130 may communicate with and/or be connected to the ultrasound device 110, the processing device 120 and/or the storage device 140. In some embodiments, the interaction with the user may be implemented through the terminal device 130. In some embodiments, the terminal device 130 may include a mobile device 131, a tablet computer 132, a notebook computer 133, etc., or any combination thereof. In some embodiments, the terminal device 130 (or all or portion of its functions) may be integrated in the processing device 120.

The storage device 140 may store data, instructions and/or any other information. In some embodiments, the storage device 140 may store the data (e.g., the ultrasound data, regurgitation information, etc.) obtained from the ultrasound device 110, the processing device 120 and/or the terminal device 130. In some embodiments, storage device 140 may store data and/or instructions that the processing device 120 executes or uses to perform the exemplary methods described in the present disclosure.

In some embodiments, the storage device 140 may include one or more storage components, and each storage component may be an independent device or a portion of other devices. In some embodiments, the storage device 140 may include a random access memory (RAM), a read only memory (ROM), a mass storage, a removable memory, a volatile read-write memory, etc., or any combination thereof. In some embodiments, the storage device 140 may be implemented on the cloud platform. In some embodiments, the storage device 140 may be a portion of the ultrasound device 110, the processing device 120 and/or the terminal device 130.

The network 150 may include any suitable network capable of facilitating an exchange of the information and/or data. In some embodiments, at least one component of the application scenario 100 of the monitoring system for regurgitation (e.g., the ultrasound device 110, the processing device 120, the terminal device 130, and the storage device 140 ) may exchange the information and/or data with at least one other components in the monitoring system for the regurgitation through the network 150. For example, the processing device 120 may obtain the ultrasonic data, etc., from the ultrasound device 110 through the network 150.

It should be noted that the above description about the application scenario 100 of the regurgitation monitoring system is provided for the purpose of illustration only, and is not intended to limit the scope of the present disclosure. For those skilled in the art, various modifications or changes may be made based on the description of the present disclosure. For example, the application scenario 100 of the regurgitation monitoring system may implement similar or different functions on other devices. However, these modifications and changes may not depart from the scope of the present disclosure.

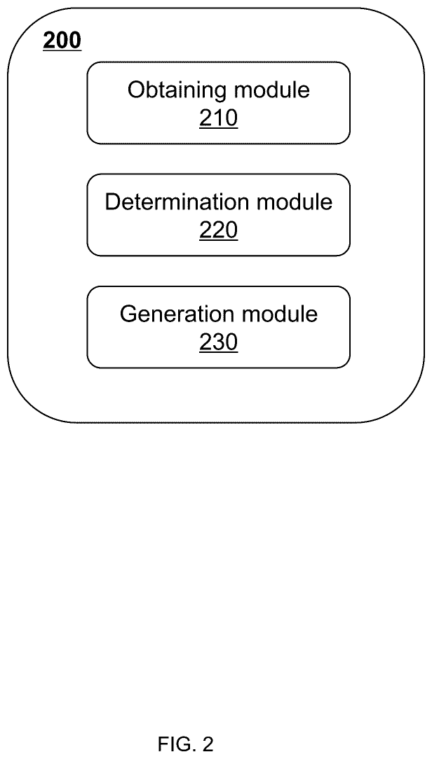

FIG. 2 is a block diagram illustrating an exemplary system 200 for monitoring a regurgitation according to some embodiments of the present disclosure.

As shown in FIG. 2, in some embodiments, the system 200 may include an obtaining module 210, a determination module 220, and a generation module 230.

The obtaining module may be configured to obtain color Doppler data of a target position. For more information about the obtaining of the color Doppler data of the target part, please refer to operation 310 and the related descriptions.

The determination module 220 may be configured to determine, based on the color Doppler data, regurgitation information of each color Doppler image of a plurality of color Doppler images. For more information on determining the regurgitation information, please refer to operation 320 and the related descriptions.

The generation module 230 may be configured to generate, based on the regurgitation information of plurality of color Doppler images, a regurgitation information data graph. The regurgitation information data graph may reflect a correspondence between at least one frame number of at least one of the plurality of color Doppler images and the regurgitation information in at least one color Doppler image. For more information on generating the regurgitation information data graph, please refer to operation 330 and the related descriptions.

It should be understood that the system and the modules of the system shown in FIG. 2 may be implemented in various ways (e.g., hardware, software, or a combination of the software and the hardware). The system and the modules of the system of the present disclosure may be implemented by a hardware circuit, which include a semiconductor such as a very large scale integration or gate array, a logic chip, a transistor, etc., or a programmable hardware device such as a field programmable gate array, a programmable logic device, etc. The system and the modules thereof of the present disclosure may be implemented by a software, for example, a software executed by various types of processors. The system and the modules thereof of the present disclosure may also be implemented by a combination of the hardware circuit and the software (e.g., a firmware).

It should be noted that the above descriptions of the system and the modules of the system are only for the convenience of description, and are used as illustrations, which do not limit the present disclosure to the scope of the illustrated embodiments. It should be understood that for those skilled in the art, after understanding the principles of the system, it is possible to combine various modules arbitrarily or form a sub-system to connect with other modules without departing from the principles.

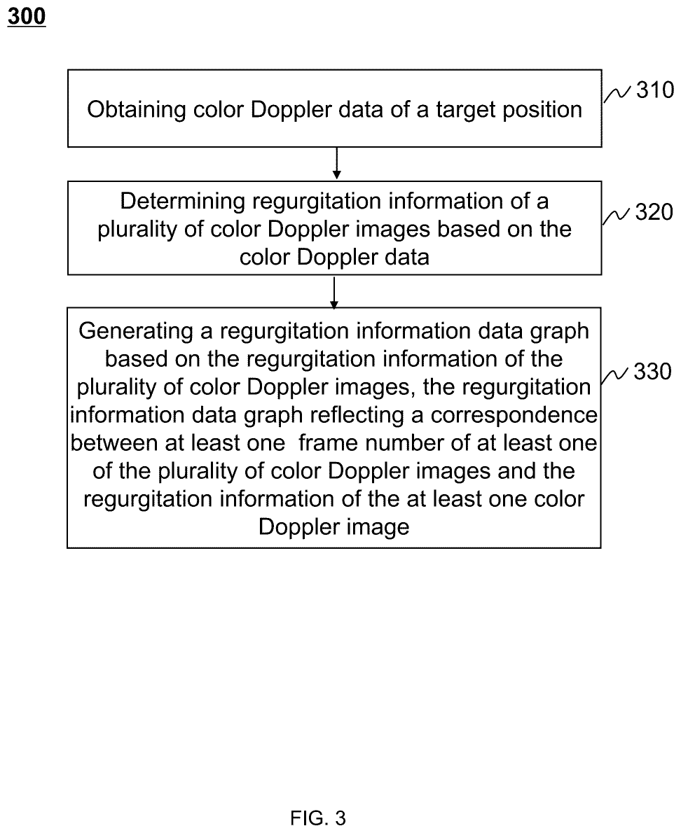

FIG. 3 is a flow chart illustrating an exemplary method for monitoring a regurgitation according to some embodiments of the present disclosure.

In 310, color Doppler data of a target position is obtained. In some embodiments, the operation 310 may be performed by the processing device 120 or the obtaining module 210.

The target position refers to a position to be imaged. In some embodiments, the target position may include a specific portion (e.g., a heart) of a body. In some embodiments, the target position may include a specific organ, such as the heart, etc.

In some embodiments, the target position includes at least one of: a tricuspid valve, a mitral valve, or an aortic valve.

The color Doppler (also known as two-dimensional Doppler) data refers to data obtained by processing echo information obtained by using a Doppler principle. In some embodiments, the color Doppler data may be displayed as a color spectrogram displayed in real time. In some embodiments, the color Doppler data may include color Doppler image data, blood flow information, etc. The blood flow information may include blood flow phase information, blood flow spectrum information, blood flow spatial information, blood flow image information (e.g., color grayscale coding information), etc.

In some embodiments, the processing device 120 may obtain the color Doppler data of the target position from the ultrasound device 110 in real time. In some embodiments, the processing device 120 may obtain the color Doppler data of the target position from the storage device 140, and a storage unit of the processing device 120, etc. In some embodiments, the processing device 120 may obtain the color Doppler data of the target position by reading from a storage device, a database, or invoking a data interface, etc.

In 320, regurgitation information of color Doppler images is determined based on the color Doppler data. In some embodiments, the operation 320 may be performed by the processing device 120 or the determination module 220.

In some embodiments, each color Doppler image of a plurality of color Doppler images is a frame of color Doppler image. In some embodiments, the plurality of color Doppler images may include all image frames in a color Doppler imaging process. In some embodiments, the plurality of color Doppler images may only include image frames with a regurgitation status. The image frames with the regurgitation status may be obtained by filtering all the image frames. For example, all image frames may be filtered based on an area of a regurgitation region in each image frame, and image frames whose area of the regurgitation region is greater than a certain threshold may be designated as the plurality of color Doppler images.

The regurgitation information refers to information reflecting the regurgitation status of a blood flow of the target position. For example, the regurgitation information may include whether there is the regurgitation, a position where the regurgitation occurs, and a blood flow volume of regurgitation, etc.

In some embodiments, the regurgitation information includes at least one of: a length of the regurgitation region, a width of the regurgitation region, an area of the regurgitation region, a direction of the blood flow in the regurgitation region, or a speed of the blood flow in the regurgitation region, etc. In some embodiments, the direction of blood flow in the regurgitation region (also referred to as a regurgitation direction) refers to a reverse direction of the direction of normal blood flow of the target position. Taking the tricuspid valve being the target position as an example, under normal circumstances, the blood flows from a right atrium through the tricuspid valve into a right ventricle, so the direction of the normal blood flow is from the right atrium to the right ventricle. At this time, the direction of blood flow in the regurgitation region may be a direction from the right ventricle to the right atrium. In some embodiments, the length of the regurgitation region refers to a maximum distance of an edge of the regurgitation region along the regurgitation direction. The width of the regurgitation region refers to a maximum distance of the edge of the regurgitation region in a direction perpendicular to the regurgitation direction. The area of the regurgitation region refers to an area occupied by the regurgitation region in the image frame. The speed of the blood flow in the regurgitation region refers to a distance per unit time during which the blood flow in the regurgitation region moves in the regurgitation direction.

In some embodiments, the processing device 120 may determine the regurgitation information of each color Doppler image in the plurality of color Doppler images through various modes. For example, by performing an image processing on the plurality of color Doppler images, the regurgitation information of the plurality of frames may be determined. Exemplarily, the processing device 120 may segment each color Doppler image according to an image segmentation process to obtain the regurgitation region. Then an image calculation process may further be performed based on the segmented regurgitation region, and the length, the width, and the area of the regurgitation region may be determined. The image segmentation process may include a threshold segmentation process, an edge detection segmentation process, an area segmentation process, a cluster segmentation process, a graph theory segmentation process, a variational equation segmentation process, a neural network segmentation process, etc. In some embodiments, regular blood flow positions (i.e., pixels) in the color Doppler image may be displayed by color blue, and positions where the regurgitation occurs may be displayed by color red. The processing device 120 may define an area displayed by color red as a regurgitation region, and further determine the length, the width, and the area of the regurgitation region, etc., based on an ultrasonic measurement process.

In some embodiments, the processing device 120 may determine the regurgitation information based on RGB values of pixels in the color Doppler image data. For example, the processing device 120 may identify pixels whose RGB values are within a red threshold range in the color Doppler image data, define the regurgitation region based on the identified pixels, and then calculate the length, the width, and the area, etc., of the regurgitation region based on a count of the identified pixels in the regurgitation region.

In some embodiments, the processing device 120 may implement a blood flow imaging of the target position based on a Doppler effect According to the Doppler effect, the ultrasound device 110 may detect different ultrasonic signals for the blood flow in different directions.

In some embodiments, the processing device 120 may determine a blood flow speed in the regurgitation region based on a Doppler frequency shift. The Doppler frequency shift refers to a difference between a receiving frequency at a receiving end and a transmitting frequency at a transmitting end. For example, the processing device 120 may determine the blood flow speed in the regurgitation region based on the following formula (1).

$$Δ f = \frac{2 v cos θ}{c} f , \qquad ( 1 )$$

where the Doppler frequency shift Δf may be obtained through f″-f, f indicates an ultrasonic frequency sent by the transmitting end, f″ indicates the frequency of a scattered echo received by the receiving end, which may be obtained by calculation; c indicates a propagation speed of the ultrasound in blood, which is about 1570 m/s; v indicates a movement speed of red blood cells, and a component of the movement speed of the red blood cells in a ultrasonic incident direction is v cos θ; and θ indicates an inclination angle of an incident wave and a scattered wave relative to the blood flow direction. In order to obtain the maximum frequency shift signal, an ultrasonic beam and the blood flow direction should form a fixed angle as much as possible, for example, θ may be equal to 50°. In this way, only v in the above formula (1) is an unknown quantity, and the blood flow speed in the regurgitation region may be obtained based on the formula (1).

In some embodiments, if v in the formula (1) is a negative value, it indicates that the blood flows away from the direction of a probe, and the corresponding frequency shift Δf may be a negative value, so the processing module 210 may also determine the direction of the blood flow based on the positive or negative of Δf.

Compared with observing the color Doppler image with naked eyes, by identifying pixels whose RGB values are within the red threshold range in the color Doppler image data, weak regurgitation signals that cannot be captured by the naked eyes may be captured, thereby making the monitoring of the regurgitation accurate.

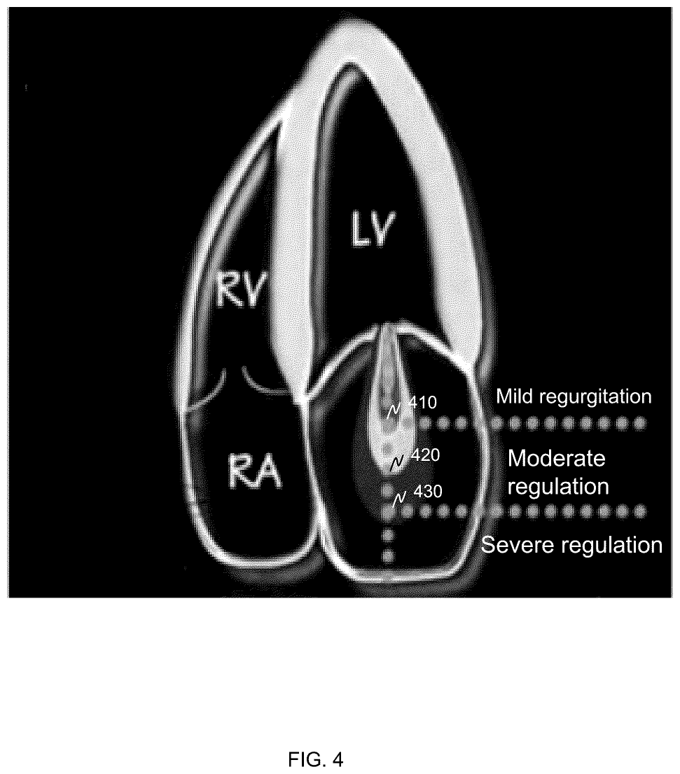

In some embodiments, the processing device 120 may determine a regurgitation degree of the target position based on the regurgitation information data graph. In some embodiments, the regurgitation degree may be a numerical value or an index reflecting a severity degree of the regurgitation of the blood flow of the target position. For example, the regurgitation degree may include a severe regurgitation, a moderate regurgitation, a mild regurgitation, etc. For contents of the regurgitation degree, please refer to FIG. 4 and the related descriptions in the present disclosure. In some embodiments, when the plurality of color Doppler images are all the color Doppler images obtained during the ultrasound imaging process, the processing device 120 may determine, based on the regurgitation information of each color Doppler image, a greatest length of the regurgitation information of the target position of the plurality of color Doppler images as a parameter for determining the regurgitation degree of the target position. In some embodiments, when the plurality of color Doppler images are all the color Doppler images obtained during the ultrasound imaging process, the processing device 120 may determine, based on the regurgitation information of each color Doppler image, the length of the regurgitation region of the target position in each color Doppler images, and the regurgitation degree of the target position may be determined based on the length of the regurgitation region of the target position in each frame. In some embodiments, when the plurality of color Doppler images are filtered image frames with the regurgitation status of the blood flow, the processing module 210 may determine, based on the regurgitation information of each filtered image frame, the regurgitation status of the target position. For example, the processing device 120 may process the length of the regurgitation region in each frame of the plurality of color Doppler images with the regurgitation status (e.g., according to an average value calculation, a weighted average calculation, etc.), and determine the regurgitation degree of the target position based on the length of the processed regurgitation region.

The regurgitation information data graph may reflect a correspondence between at least one frame number of at least one of the plurality of color Doppler images and the regurgitation information of the at least one color Doppler image. For a structure, a content, a manifestation, and a generation mode, please refer to descriptions in operation 330.

In some embodiments, the processing device 120 may generate a regurgitation warning signal based on the regurgitation degree of the target position. The regurgitation warning signal may be a prompt reflecting the regurgitation degree of the blood flow of the target position. In some embodiments, the processing device 120 may generate the regurgitation warning signal based on the regurgitation degree of the target position after the ultrasound imaging process. In some embodiments, the processing device 120 may generate the regurgitation warning signal for each color Doppler image in real time based on the regurgitation degree of the target position of the frame after each color Doppler image is obtained.

In some embodiments, the processing device 120 may determine the length of the regurgitation region of the target position based on the regurgitation information of each color Doppler image in the plurality of color Doppler images. For example, the processing device 120 may determine pixels in the color Doppler images whose RGB values are within the red threshold range, define the regurgitation region based on the pixels, and then calculate, based on the count of the pixels in the regurgitation region along the regurgitation direction, the length of the regurgitation region. In some embodiments, the processing device 120 may define the area displayed in red as the regurgitation region, and further determine the length of the regurgitation region of the target position through the ultrasound measurement.

In some embodiments, the length of the regurgitation region of the target position may be the length of the longest regurgitation region among the plurality of color Doppler images. For example, when the plurality of color Doppler images are all the color Doppler images obtained during the ultrasound imaging process, the processing device 120 may designate the length of longest regurgitation region among the plurality of color Doppler images as the length of the regurgitation region of the target position based on the regurgitation information of each color Doppler image.

In some embodiments, the length of the regurgitation region of the target position may be an average value or a weighted average value of the lengths of the regurgitation regions in each color Doppler image of the plurality of color Doppler images. For example, when the plurality of color Doppler images may be filtered image frames with the blood flow regurgitation, the processing device 120 may process (e.g., according to an average value calculation, a weighted average calculation, etc.) the length of the regurgitation region in each frame of the plurality of color Doppler images with the regurgitation status, and designate the processed length of the regurgitation region as the length of the regurgitation region of the target position.

In some embodiments, the processing device 120 may determine, based on the length of the regurgitation region and the length of the target position along the regurgitation direction, the regurgitation degree of the target position. The length of the target position along the regurgitation direction refers to a maximum distance of an edge of the target position along the regurgitation direction, for example, a maximum distance of edges of a mitral valve along the regurgitation direction. For example, the processing device 120 may determine the regurgitation degree of the target position based on a ratio of the length of the regurgitation region of the target position to the length of the target position along the regurgitation direction.

In some embodiments, when the ratio of the length of the regurgitation region of the target position to the length of the target position along the regurgitation direction is less than ⅓, the processing device 120 may determine that the regurgitation degree of the target position is a mild regurgitation. In some embodiments, when the ratio of the length of the regurgitation region of the target position to the length of the target position along the regurgitation direction is in a range of ⅓-⅔, inclusive of ⅓ and ½, the processing device 120 may determine that the regurgitation degree of the target position is a moderate regurgitation. In some embodiments, when the ratio of the length of the regurgitation region of the target position to the length of the target position along the regurgitation direction is greater than ⅔, the processing device 120 may determine that the regurgitation degree of the target position is a severe regurgitation.

FIG. 4 is a schematic diagram illustrating a mitral valve regurgitation based on other embodiments of the present disclosure. For example, in the schematic diagram of the mitral valve regurgitation shown in FIG. 4, when the regurgitation region reaches a horizontal position 410 of a valve ring, the length of the regurgitation region reaches ⅓ of a left atrium, and the regurgitation degree is the mild regurgitation. Similarly, when the regurgitation region reaches a middle position 420 of the left atrium, the length of the regurgitation region reaches ½ of the left atrium, and the regurgitation degree is the moderate regurgitation. When the regurgitation region reaches a top position 430 of the left atrium, the length of the regurgitation region exceeds ⅔ of the left atrium, and the regurgitation degree is the severe regurgitation.

It should be understood that the determining the regurgitation degree described above is only for illustration, and the processing device 120 may also determine the regurgitation degree based on other regurgitation information (e.g., an area of the regurgitation region, a width of the regurgitation region, a speed of regurgitation, etc.). For example, when the area of the regurgitation region is less than 3 cm² or a ratio value of the area of the regurgitation region to the area of the target position is less than 20%, the processing device 120 may determine that the regurgitation degree of the target position is the mild regurgitation. Exemplarily, when monitoring a regurgitation degree of a tricuspid valve, the target position may be a tricuspid valve orifice; when monitoring a regurgitation degree of a mitral valve, the target position may be a mitral valve orifice; and when monitoring a regurgitation degree of a pulmonary valve, the target position may be a pulmonary valve orifice. For another example, when the area of the regurgitation region is within a range of 3 cm²-4.5 cm², or the ratio value of the area of the regurgitation region to the area of the target position is in a range of 20%-50%, inclusive of 20% and 50%, the processing device 120 may determine that the regurgitation degree of the target position is the moderate regurgitation. The area of the target position refers to an area occupied by the target position in an image frame, for example, the area occupied by the mitral valve in the image frame. When the area of the regurgitation region is greater than 4.5 cm² or the ratio of the area of the regurgitation region to the area of the target position is greater than 50%, the processing device 120 may determine that the regurgitation degree of the target position is the severe regurgitation. For another example, when the regurgitation speed is less than 150 cm/s, the processing device 120 may determine that the regurgitation degree of the target position is the mild regurgitation; when the regurgitation velocity is in a range of 150 cm/s-450 cm/s, inclusive of 150 cm/s and 450 cm/s, the processing device 120 may determine that the regurgitation degree of the target position is the moderate regurgitation; and when the regurgitation velocity is greater than 450 cm/s, the processing device 120 may determine that the regurgitation degree of the target position is the severe regurgitation. For another example, when the length of the regurgitation region is less than 20 mm, the processing device 120 may determine that the regurgitation degree of the target position is the mild regurgitation; when the length of the regurgitation region is within a range of 20 mm-45 mm, inclusive of 20 mm and 45 mm, the processing device 120 may determine that the regurgitation degree of the target position is the moderate regurgitation; and when the length of the regurgitation region is greater than 45 mm, the processing device 120 may determine that the regurgitation degree of the target position is the severe regurgitation. For another example, when a proportion of the width of the regurgitation region (that is, a ratio of the width of the regurgitation region to an inner diameter of the target position) is in a range of 20%-40%, the processing device 120 may determine that the regurgitation degree of the target position is the mild regurgitation; when the proportion of the width of the regurgitation region is in a range of 40%-60%, inclusive of 40% and 60%, the processing device 120 may determine that the regurgitation degree of the target position is the moderate regurgitation; and when the proportion of the width of the regurgitation region is greater than 60%, the processing device 120 may determine that the regurgitation degree of the target position is the severe regurgitation. For another example, when a regurgitation pressure half-drop time is greater than 400 ms, the processing device 120 may determine that the regurgitation degree of the target position is the mild regurgitation; when the regurgitation pressure half-drop time is in a range of 250 ms-400 ms, inclusive of 250 ms and 400 ms, the processing device 120 may determine that the regurgitation degree of the target position is the moderate regurgitation; and when the regurgitation pressure half-drop time is less than 250 ms, the processing device 120 may determine that the regurgitation degree of the target position is the severe regurgitation.

In some embodiments, the processing device 120 may obtain a plurality of results of the regurgitation degree of the target position based on various regurgitation information, and combine the plurality of results to obtain the regurgitation degree of the target position. For example, if a counts of different results among the plurality of results are the same, the result with a higher regurgitation degree may be designated as the regurgitation degree of the target position. Exemplarily, if the regurgitation degree of the target position based on the width of the regurgitation region is the mild regurgitation, and the regurgitation degree of the target position based on the area of the regurgitation region is the moderate regurgitation, combining the results of the regurgitation degrees of the above two target portions, the regurgitation degree of the target portion may be determined as the moderate regurgitation. For another example, if the counts of different results among the plurality of results are different, the result with the greatest count may be designated as the regurgitation degree of the target position. For example, if the regurgitation degree of the target position obtained based on the length of the regurgitation region and the width of the regurgitation region is the mild regurgitation, and the regurgitation degree of the target position obtained based on the regurgitation speed of the regurgitation region is the moderate regurgitation, combining the results of the regurgitation degrees of the above three target portions, the regurgitation degree of the target portion may be determined as the mild regurgitation.

In some embodiments, the processing device 120 may determine the regurgitation degree of the target position based on a trained machine learning model and the regurgitation information data graph. For example, the processing device 120 may input the regurgitation information data graph into the trained machine learning model, and the trained machine learning model may output the regurgitation degree of the target position. Through training, the machine learning model may learn, based on features of the regurgitation information data graph (e.g., the length of the regurgitation region, the area of the regurgitation region, the speed of the regurgitation region, and the direction of blood flow in the regurgitation region, etc.), the relationship between the degree of regurgitation and the features, thereby obtaining the evaluation results of the regurgitation degree.

An input of the trained machine learning model may include the regurgitation information data graph (e.g., the regurgitation information data graph in the form of a line graph, a curve graph, histograms, a pie chart, etc., corresponding to target position) corresponding to a target position of an object. An output of the trained machine learning model may include the regurgitation degree of the target position.

In some embodiments, the processing device 120 may train an initial machine learning model based on a great count of training samples, so as to update parameters of the machine learning model to obtain the trained machine learning model. In some embodiments, each of the at least a portion of the training samples includes a sample regurgitation information data graph, and the regurgitation information of a plurality of groups of samples of the plurality of color Doppler images corresponding to the sample regurgitation information data graph. A group of sample regurgitation information corresponding to a certain frame of color Doppler images may include: at least one of the lengths of the regurgitation region, the width of the regurgitation region, the area of the regurgitation region, the direction of blood flow in the regurgitation region, or the speed of blood flow in the regurgitation region of the color Doppler image frame. The sample label may be a standard regurgitation degree, e.g., the mild regurgitation, the moderate regurgitation, the severe regurgitation, etc. In some embodiments, the sample label may be obtained by manual labeling.

In some embodiments, the trained machine learning model may be constructed based on a sequence model. For example, a typical sequence model may include a recurrent neural network (RNN), a long short-term memory network (LSTM), or any combination thereof. As another example, the machine learning model may include a convolutional neural network, a recurrent neural network, and a fully connected layer. In some embodiments, the convolutional neural network may separately process the plurality of groups of sample regurgitation information, and extract features of each group of sample regurgitation information. A size of a convolution kernel may be set according to experience or requirements, for example, the size of the convolution kernel may be 3*3. In some embodiments, the sequence model may process the features of the regurgitation information of these samples to extract sequence features. The sequence features may include feature relationships between previous and subsequent frames. In some embodiments, the convolutional neural network may also process the sample regurgitation information data graph to extract image features of the sample regurgitation information data graph. In some embodiments, the fully connected layer may process the sequence features and/or the image features to determine the regurgitation degree of the target position.

In some embodiments, the trained machine learning model may be constructed based on a model, and the model may include a support vector machine model, a Logistic regression model, a naive Bayesian classification model, a Gaussian distribution Bayesian classification model, a decision tree model, a random forest model, a k-nearest neighbor (KNN) classification model, a neural network model, etc.

In some embodiments, the processing device 120 may adjust parameters of the machine learning model to reduce a difference between a predicted regurgitation degree and a regurgitation degree label.

In some embodiments, the processing device 120 may reflect the difference between the predicted regurgitation degree and the regurgitation degree label by constructing a loss function. The loss function may include a cross-entropy loss function, a mean square error loss function, an exponential loss function, a logarithmic loss function, and a square loss function, etc.

In some embodiments, the processing device 120 may perform several iterative trainings on the initial machine learning model on a training set to obtain the trained machine learning model. A mode for the iterative training may include: calculating a gradient of the loss function, and iteratively updating the parameters of the machine learning model through a gradient descent method, so as to reduce the difference between the predicted regurgitation degree and the regurgitation degree label. The gradient descent method may include a standard gradient descent method and a stochastic gradient descent method, etc. A variety of learning rate decay strategies may be used in the iterative training, such as a segmental decay, a reverse time decay, an exponential decay, and an adaptive decay. When the iteration termination condition is satisfied, the iterative training may be terminated. The iteration termination condition may include that the loss function converges or is less than a preset threshold, a count of iterations reaches a preset count, etc.

In some embodiments, the processing module 210 may issue a regurgitation warning signal based on the regurgitation degree of the target position. The warning signal may be in a form of a text, a voice, an image, a pop-up window, an indicator light, etc., and any combination thereof. For example, when the regurgitation degree of the target position is the mild regurgitation, the mild regurgitation may be prompted through the text and/or the voice. For another example, when the regurgitation degree of the target position is the moderate regurgitation, the moderate regurgitation may be prompted through the text, the voice and/or the pop-up window. For another example, when the regurgitation degree of the target position is the severe regurgitation, the severe regurgitation may be prompted through the text, the voice, the pop-up windows and/or the indicator lights.

In 330, a regurgitation information data graph is generated based on the regurgitation information of the plurality of color Doppler images. In some embodiments, the regurgitation information data graph may reflect a correspondence between at least one frame number of at least one of the plurality of color Doppler images and the regurgitation information of the at least one color Doppler image. In some embodiments, the operation 330 may be performed by the processing device 120 or the generation module 230.

In some embodiments, the regurgitation information data graph may include a line graph, a curve graph, a histogram, a pie graph, etc. and any combination thereof. In some embodiments, an abscissa of the regurgitation information data graph may be the frame number of the color Doppler images. In some embodiments, an ordinate of the regurgitation information data graph may include one or more items of the regurgitation information. For example, the ordinate may only include the length of the regurgitation region, or the ordinate may include both the length of the regurgitation region and the area of the regurgitation region. In some embodiments, a content included in the ordinate of the regurgitation information data graph may be set by the system based on requirements and/or experience. In some embodiments, the content included in the ordinate of the regurgitation information data graph may be set by the user.

In some embodiments, the generation module 230 may generate the regurgitation information data graph in real time based on the regurgitation information of each color Doppler image. Compared with the condition of the existing ultrasonic imaging process, in which the operator (e.g., a physician) needs to manually select a color Doppler image for data measurement based on the visual observation of the regurgitation situation, the real-time generation of the regurgitation information data graph described in some embodiments of the present disclosure may make the regurgitation data more automatically and visually display the blood flow regurgitation of the target position in real time, thereby making the working process of the ultrasound imaging process smooth.

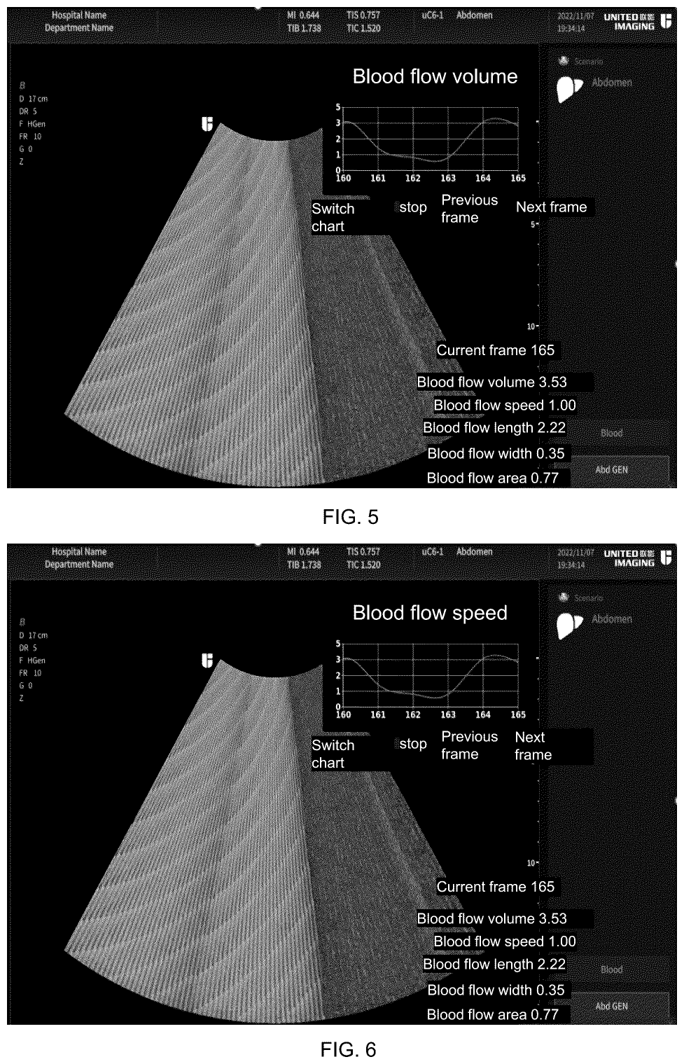

FIG. 5 is a schematic diagram illustrating an exemplary ultrasound imaging operation interface according to some embodiments of the present disclosure. FIG. 6 is a schematic diagram illustrating an exemplary ultrasound imaging operation interface according to some embodiments of the present disclosure. As shown in FIG. 5, in a process of performing an imaging at a target position (e.g., a heart), an operator (e.g., a physician) does not need to manually stop on an interface displaying a certain frame. An ultrasound imaging operation interface may display a real-time color Doppler image of a target position (example is made with black and white image only), a frame number (e.g., the 165th frame), regurgitation information, and a blood flow curve that reflects a relationship between a blood flow and the frame number (that is, a regurgitation information data graph). In the example of the real-time display of regurgitation information on the ultrasound imaging operation interface as shown in FIG. 5, a blood flow volume of the regurgitation (that is, the “blood flow volume” shown in the figure) is 3.53 ml/s, a regurgitation speed (that is, “blood flow speed” shown in the figure) is 1.00 m/s, a length of the regurgitation region (that is, “blood flow length” shown in the figure) is 2.22 cm, the width of the regurgitation region (that is, “blood flow width” shown in the figure) is 0.35 cm, and the area of the regurgitation region (that is, “blood flow area” shown in the figure) is 0.77 cm². As shown in FIG. 5, the blood flow volume graph reflecting the relationship between the blood flow volume and the frame number may show a magnitude of the blood flow in the plurality of Doppler frames before the current frame in real time. When the operator clicks a “switch chart” button, FIG. 5 jumps to FIG. 6, and the blood flow volume curve reflecting the relationship between the blood flow volume and the frame number may be converted into a blood flow speed curve reflecting the regurgitation speed. As shown in FIG. 6, the Doppler image and the regurgitation information displayed on the ultrasound imaging operation interface may be consistent with those in FIG. 5, and the difference lies in the content displayed on the graph. In some embodiments, as shown in FIG. 5 and FIG. 6, the operator may click a “stop” button during the imaging process to stop the Doppler image and the curve on the operation interface at a certain frame, while still obtaining the Doppler image.

In some embodiments, based on the user's interaction with the regurgitation information data graph, the generation module 230 may display the color Doppler image of the target position and/or the regurgitation information corresponding to the color Doppler image. For example, the operator may click a “previous frame” or a “next frame” button below the regurgitation information data graph, so that the interface displays the image of a certain frame and the related regurgitation information. For another example, in some embodiments, the operator may click a certain frame node (e.g., the node of the 165th frame) in the regurgitation information data graph. At this time, as shown in FIG. 6, the gurgitation information corresponding to the 165th color Doppel frame is displayed at a bottom right corner of the interface. The regurgitation information includes the blood flow volume, the blood flow speed, the length of the regurgitation region, the width of the regurgitation region, the area of the regurgitation region, the regurgitation degree, etc. The middle portion shows 165 frames of the color Doppler images.

Some embodiments of the present disclosure may (1) automatically identify the regurgitation status of the heart valve in real time, for example, effectively identify the regurgitation status of the tricuspid valve, the mitral valve, and the aortic valve, as well as a pathological regurgitation caused by an atrioventricular septal defect; (2) detect extremely weak regurgitation signals to make the detection accurate; (3) display the real-time data in the form of a line graph/a curve graph, etc., so that the users may accurately locate a key frames based on the real-time data to improve the detection efficiency; (4) open a key frame preview by clicking a node in the line graph/the curve graph, and the key frame may visually display a regurgitation region of interest (ROI) area, the length, the width, the area, the blood flow direction, the speed, etc.; and (5) automatically broadcast the warning for the regurgitation signals, and perform an internal calculation on the stored data to achieve a quantitative analysis, evaluate the severities of the valvular regurgitation, the regurgitation of the atrioventricular septal defect, etc., and perform a medical classification (the mild regurgitation, the moderate regurgitation, and the severe regurgitation), so as to provide doctors with the basis for a clinical diagnosis.

The basic concepts have been described above, apparently, for those skilled in the art, the above-mentioned detailed disclosure is only used as an example, and it does not constitute a limitation of the present disclosure. Although not expressly stated here, those skilled in the art may make various modifications, improvements, and corrections to the present disclosure. These alterations, improvements, and modifications are intended to be suggested by this disclosure, and are within the spirit and scope of the exemplary embodiments of the present disclosure.

At the same time, the present disclosure uses specific words to describe the embodiments of the present disclosure. As “one embodiment,” “an embodiment,” and/or “some embodiments” means a certain feature, structure, or characteristic of the present disclosure at least one embodiment. Therefore, it is emphasized and should be appreciated that two or more references to “an embodiment,” or “one embodiment,” or “an alternative embodiment” in various portions of the present disclosure are not necessarily all referring to the same embodiment. In addition, some features, structures, or features in the present disclosure of one or more embodiments may be appropriately combined.

In addition, unless explicitly stated in the claims, the order of processing elements and sequences described in the present disclosure, the use of counts and letters, or the use of other names are not configured to limit the sequence of processes and methods in the present disclosure. While the above disclosure discusses by way of various examples some embodiments of the invention that are presently believed to be useful, it is to be understood that such details are for purposes of illustration only and that the appended claims are not limited to the disclosed embodiments. Rather, the claims aim to cover all corrections and equivalent combinations that are in line with the nature and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the counts expressing quantities, properties, and so forth, used to describe and claim certain embodiments of the application are to be understood as being modified in some instances by the term “about,” “approximate,” or “substantially.” Unless otherwise stated, the “about,” “approximately,” or “substantially” indicates that the stated figure allows for a variation of ±20%. Accordingly, in some embodiments, the numerical parameters used in the present disclosure and claims are approximations that can vary depending upon the desired characteristics of individual embodiments. In some embodiments, the numerical parameters should consider the effective digits specified and use a general digit reservation method. Although in some embodiments of the present disclosure, the numerical domain and parameters used to confirm the range of its scope are approximate values , the setting of such values may be as precise as possible within the feasible range in specific embodiments.

For each patent, patent application, patent application publications and other materials cited by the present disclosure, such as articles, books, instructions, publications, documents, etc., all of them will be incorporated in the present disclosure as a reference. History application documents that are inconsistent or conflictive with the contents of the present disclosure are excluded, as well as documents (currently or subsequently appended to the present disclosure) limiting the broadest scope of the claims of the present disclosure. It should be noted that, if there is any inconsistency or conflict between the descriptions, definitions, and/or usage of terms in subsidiary information of the present disclosure and the contents of the present disclosure, the descriptions, definitions, and/or usage of terms in the present disclosure shall prevail.

Finally, it is to be understood that the embodiments of the application disclosed herein are illustrative of the principles of the embodiments of the present disclosure. Other deformation may also belong to the scope of the present disclosure. Therefore, as an example rather than restrictions, the replacement configuration of the embodiment of this disclosure may be consistent with the teaching of the present disclosure. Accordingly, the embodiments of the present disclosure are not limited to the embodiments introduced and described in the present disclosure explicitly.

What is claimed is:

1. A system for monitoring regurgitation comprising:
at least one storage device including a set of instructions; and
at least one processor in communication with the at least one storage device, wherein when executing the set of instructions, the at least one processor is directed to cause the system to perform operations including:determining, by the at least one processor, regurgitation information of a plurality of color Doppler images based on color Doppler data of a target position; and
generating, by the at least one processor, a regurgitation information data graph based on the regurgitation information of the plurality of color Doppler images, the regurgitation information data graph reflecting a correspondence between at least one frame number of at least one of the plurality of color Doppler images and the regurgitation information of the at least one color Doppler image.

2. The system of claim 1, wherein the operations further comprise:
determining, by the at least one processor, a regurgitation degree of the target position based on the regurgitation information data graph.

3. The system of claim 2, wherein determining, the by the at least one processor, the regurgitation degree of the target position based on the regurgitation information data graph comprises:
determining, by the at least one processor, the regurgitation degree of the target position based on a maximum value, an average value, or a weighted value of the regurgitation information in the regurgitation information data graph.

4. The system of claim 2, wherein the determining, by the at least one processor, the regurgitation degree of the target position based on the regurgitation information data graph comprises:
determining, by the at least one processor, a length of a regurgitation region of the target position based on the regurgitation information of the plurality of color Doppler images; and
determining, by the at least one processor, the regurgitation degree of the target position based on a ratio of the length of the regurgitation region of the target position to a length of the target position along a regurgitation direction.

5. The system of claim 4, wherein determining, the by the at least one processor, the regurgitation degree of the target position based on the ratio of the length of the regurgitation region of the target position to the length of the target position along the regurgitation direction comprises:
in response to determining that a ratio of the length of the regurgitation region of the target position to the length of the target position along the regurgitation direction is less than ⅓, determining, by the at least one processor, that the regurgitation degree of the target position is a mild regurgitation;
in response to determining that the ratio value of the length of the regurgitation region of the target position to the length of the target position along the regurgitation direction is in a range of ⅓-⅔, determining, by the at least one processor, that the regurgitation degree of the target position is a moderate regurgitation; and
in response to determining that the ratio value of the length of the regurgitation region of the target position to the length of the target position along the regurgitation direction is greater than ⅔, determining, by the at least one processor, that the regurgitation degree of the target position is a severe regurgitation.

6. The system of claim 2, wherein the determining, by the at least one processor, the regurgitation degree of the target position based on the regurgitation information data graph comprises:
determining, by the at least one processor, a width of a regurgitation region of the target position based on the regurgitation information of the plurality of color Doppler images; and
determining, by the at least one processor, the regurgitation degree of the target position based on a ratio of the width of the regurgitation region of the target position to an inner diameter of the target position.

7. The system of claim 6, wherein the determining, by the at least one processor, the regurgitation degree of the target position based on the ratio of the width of the regurgitation region of the target position to the inner diameter of the target position comprises:
in response to determining that the ratio of the width of the regurgitation region of the target position to the inner diameter of the target position is in a range of 20%-40%, determining, by the at least one processor, that the regurgitation degree of the target position is a mild regurgitation;
in response to determining that the ratio of the width of the regurgitation region of the target position to the inner diameter of the target position is in a range of 40%-60%, determining, by the at least one processor, that the regurgitation degree of the target position is a moderate regurgitation; or
in response to determining that the ratio of the width of the regurgitation region of the target position to the inner diameter of the target position is greater than 60%, determining, by the at least one processor, that the regurgitation degree of the target position is a severe regurgitation.

8. The system of claim 2, wherein the determining, by the at least one processor, the regurgitation degree of the target position based on the regurgitation information data graph comprises:
determining, by the at least one processor, an area of a regurgitation region of the target position based on the regurgitation information of the plurality of color Doppler images; and
determining, by the at least one processor, the regurgitation degree of the target position based on the area of the regurgitation region of the target position and an area of the target position.

9. The system of claim 8, wherein the determining, by the at least one processor, the regurgitation degree of the target position based on the area of the regurgitation region of the target position and the area of the target position comprises:
in response to determining that the area of the regurgitation region is less than 3 cm² or a ratio of the area of the regurgitation region to the area of the target position is less than 20%, determining, by the at least one processor, that the regurgitation degree of the target position is a mild regurgitation;
in response to determining that the area of the regurgitation region is in the range of 3 cm²-4.5 cm² or the ratio of the area of the regurgitation region to the area of the target position being in the range of 20%-50%, determining, by the at least one processor, that the regurgitation degree of the target position is a moderate regurgitation; and
in response to determining that the area of the regurgitation region is greater than 4.5 cm² or the ratio of the area of the regurgitation region to the area of the target position is greater than 50%, determining, by the at least one processor, that the regurgitation degree of the target position is a severe regurgitation.

10. The system of claim 1, wherein the determining, by the at least one processor, the regurgitation degree of the target position based on the regurgitation information data graph comprises:
determining, by the at least one processor, a regurgitation speed of the target position based on the regurgitation information of the plurality of color Doppler images; and
determining, by the at least one processor, the regurgitation degree of the target position based on the regurgitation speed.

11. The system of claim 10, wherein the determining, by the at least one processor, the regurgitation degree of the target position based on the regurgitation speed comprises:
in response to determining that the regurgitation speed is less than 150 cm/s, determining, by the at least one processor, that the regurgitation degree of the target position is a mild regurgitation;
in response to determining that the regurgitation speed is in a range of 150 cm/s-450 cm/s, determining, by the at least one processor, that the regurgitation degree of the target position is a moderate regurgitation; and
in response to determining that the regurgitation speed is greater than 450 cm/s, determining, by the at least one processor, that the regurgitation degree of the target position a severe regurgitation.

12. The system of claim 2, wherein, the operations further comprise:
generating, by the at least one processor, a regurgitation warning signal based on the regurgitation degree of the target position.

13. The system of claim 1, wherein the operations further comprise: displaying, by the at least one processor, a color Doppler image of the target position according to an interaction between a user and the regurgitation information data graph, regurgitation information of the color Doppler image being displayed on the color Doppler image.

14. The system of claim 1, wherein the operations further comprise: displaying, one of the at least one of the plurality of color Doppler images and the regurgitation information data graph simultaneously in the user interface.

15. The system of claim 1, wherein the regurgitation information comprises at least one of:
a length of a regurgitation region,
a width of the regurgitation region,
an area of the regurgitation region,
a direction of blood flow in the regurgitation region, or
a speed of blood flow in the regurgitation region.

16. The system of claim 1, wherein the regurgitation information data graph is obtained based on one or more color Doppler images selected from the plurality of color Doppler images, in each of the one or more color Doppler images, the regurgitation information is greater than a threshold.

17. The system of claim 1, wherein the target position includes at least one of: a tricuspid valve, a mitral valve, or an aortic valve.

18. A method for monitoring regurgitation implemented on a computing device having at least one processor and at least one computer-readable storage medium, the method comprising:determining, by the at least one processor, regurgitation information of a plurality of color Doppler images based on color Doppler data of a target position; and
generating, by the at least one processor, a regurgitation information data graph based on the regurgitation information of the plurality of color Doppler image, the regurgitation information data graph reflecting a correspondence between at least one frame number of at least one of the plurality of color Doppler images and the regurgitation information of the at least one color Doppler image.

19. The method of claim 18 further comprising:
determining, by the at least one processor, a regurgitation degree of the target position based on the regurgitation information data graph.

20. A non-transitory computer readable storage medium, wherein the storage medium stores computer instructions, when the computer reads the computer instructions, the computer implements a method, the method comprising:determining, by the at least one processor, regurgitation information of a plurality of color Doppler images based on color Doppler data of a target position; and
generating, by the at least one processor, a regurgitation information data graph based on the regurgitation information of the plurality of color Doppler images, the regurgitation information data graph reflecting a correspondence between at least one frame number of at least one of the plurality of color Doppler images and the regurgitation information of the at least one color Doppler image.
